SEQUENCE LISTING (40 sequences)

SEQ ID NO: 1 - protein, 11 aa, synthetic construct
  REGION 1..11: note = Synthetic: BDC2.5 HIP: C-Peptide/ChrA-WE14
LQTLALWSRMD

SEQ ID NO: 2 - protein, 14 aa, synthetic construct
  REGION 1..14: note = Synthetic: Chromogranin A WE14 peptide
WSRMDQLAKELTAE

SEQ ID NO: 3 - protein, 110 aa, Mus musculus
  REGION 1..110: note = misc_feature - C-Peptide peptide (mouse)
MALWMRFLPLLALLFLWESHPTQAFVKQHLCGSHLVEALYLVCGERGFFYTPMSRREVEDPQVAQLELGGGPGAGDLQTLALEVAQQKRGIVDQCCTSICSLYQLENYCN

SEQ ID NO: 4 - protein, 14 aa, Homo sapiens
  REGION 1..14: note = misc_feature - Human HIP11
SLQPLALEAEDLQV

SEQ ID NO: 5 - protein, 31 aa, Homo sapiens
  REGION 1..31: note = misc_feature - C-peptide peptide (human)
EAEDLQVGQVELGGGPGAGSLQPLALEGSLQ

SEQ ID NO: 6 - protein, 7 aa, Homo sapiens
  REGION 1..7: note = misc_feature - C-terminal sequence from HIP11
SLQPLAL

SEQ ID NO: 7 - protein, 7 aa, Homo sapiens
  REGION 1..7: note = misc_feature - N-terminal sequence from HIP11
EAEDLQV

SEQ ID NO: 8 - protein, 15 aa, synthetic construct
  REGION 1..15: note = Synthetic: HIP1
GQVELGGWSKMDQLA

SEQ ID NO: 9 - protein, 15 aa, synthetic construct
  REGION 1..15: note = Synthetic: HIP2
GQVELGGLEGQEEEE

SEQ ID NO: 10 - protein, 15 aa, synthetic construct
  REGION 1..15: note = Synthetic: HIP3
GQVELGGGEAEDLQV

SEQ ID NO: 11 - protein, 15 aa, synthetic construct
  REGION 1..15: note = Synthetic: HIP4
GQVELGGGGIVEQCC

SEQ ID NO: 12 - protein, 15 aa, synthetic construct
  REGION 1..15: note = Synthetic: HIP5
GQVELGGGTPIESHQ

SEQ ID NO: 13 - protein, 15 aa, synthetic construct
  REGION 1..15: note = Synthetic: HIP6
GQVELGGGNAVEVLK

SEQ ID NO: 14 - protein, 15 aa, synthetic construct
  REGION 1..15: note = Synthetic: HIP7
GQVELGGGFLGEGHH

SEQ ID NO: 15 - protein, 15 aa, synthetic construct
  REGION 1..15: note = Synthetic: HIP8
GQVELGGGSSPETLI

SEQ ID NO: 16 - protein, 14 aa, synthetic construct
  REGION 1..14: note = Synthetic: HIP9
SLQPLALWSKMDQL

SEQ ID NO: 17 - protein, 14 aa, synthetic construct
  REGION 1..14: note = Synthetic: HIP10
SLQPLALLEGQEEE

SEQ ID NO: 18 - protein, 14 aa, synthetic construct
  REGION 1..14: note = Synthetic: HIP12
SLQPLALGIVEQCC

SEQ ID NO: 19 - protein, 14 aa, synthetic construct
  REGION 1..14: note = Synthetic: HIP13
SLQPLALTPIESHQ

SEQ ID NO: 20 - protein, 14 aa, synthetic construct
  REGION 1..14: note = Synthetic: HIP14
SLQPLALNAVEVLK

SEQ ID NO: 21 - protein, 14 aa, synthetic construct
  REGION 1..14: note = Synthetic: HIP15
SLQPLALFLGEGHH

SEQ ID NO: 22 - protein, 14 aa, synthetic construct
  REGION 1..14: note = Synthetic: HIP16
SLQPLALSSPETLI

SEQ ID NO: 23 - protein, 14 aa, synthetic construct
  REGION 1..14: note = Synthetic: insB:9-23
SHLVEALYLVCGER

SEQ ID NO: 24 - protein, 14 aa, synthetic construct
  REGION 1..14: note = Synthetic: insB:9-23R22E
SHLVEALYLVCGEE

SEQ ID NO: 25 - protein, 16 aa, synthetic construct
  REGION 1..16: note = Synthetic: ins64 79
GQVELGGGPGAGSLQP

SEQ ID NO: 26 - protein, 16 aa, synthetic construct
  REGION 1..16: note = Synthetic: ins75 90
GSLQPLALEGSLQKRG

SEQ ID NO: 27 - protein, 16 aa, synthetic construct
  REGION 1..16: note = Synthetic: ChgA334 349
KEWEDSKRWSKMDQLA

SEQ ID NO: 28 - protein, 16 aa, synthetic construct
  REGION 1..16: note = Synthetic: ChgA350 365
KELTAEKRLEGQEEEE

SEQ ID NO: 29 - protein, 16 aa, synthetic construct
  REGION 1..16: note = Synthetic: Ins49 64
FYTPKTRREAEDLQVG

SEQ ID NO: 30 - protein, 16 aa, synthetic construct
  REGION 1..16: note = Synthetic: Ins82 97
LEGSLQKRGIVEQCCT

SEQ ID NO: 31 - protein, 16 aa, synthetic construct
  REGION 1..16: note = Synthetic: IAPP15 30
VALNHLKATPIESHQV

SEQ ID NO: 32 - protein, 16 aa, synthetic construct
  REGION 1..16: note = Synthetic: IAPP66 81
GSNTYGKRNAVEVLKR

SEQ ID NO: 33 - protein, 16 aa, synthetic construct
  REGION 1..16: note = Synthetic: ScG1432 447
SDTREEKRFLGEGHHR

SEQ ID NO: 34 - protein, 16 aa, synthetic construct
  REGION 1..16: note = Synthetic: NP-Y60 75
TRQRYGKRSSPETLIS

SEQ ID NO: 35 - protein, 20 aa, synthetic construct
  REGION 1..20: note = Synthetic: Insulin specific peptide
EAEDLQVGQVELGGGPGAGS

SEQ ID NO: 36 - protein, 13 aa, synthetic construct
  REGION 1..13: note = Synthetic: Insulin specific peptide
GSLQPLALEGSLQ

SEQ ID NO: 37 - protein, 17 aa, synthetic construct
  REGION 1..17: note = Synthetic: Insulin specific peptide
GPGAGSLQPLALEGSLQ

SEQ ID NO: 38 - protein, 20 aa, synthetic construct
  REGION 1..20: note = Synthetic: Insulin specific peptide
EAEDLQVGQVELGGGPGAGS

SEQ ID NO: 39 - protein, 22 aa, synthetic construct
  REGION 1..22: note = Synthetic: Insulin specific peptide
EAEDLQVGQVELGGGPGAGSLQ

SEQ ID NO: 40 - protein, 19 aa, synthetic construct
  REGION 1..19: note = Synthetic: Insulin specific peptide
GGGPGAGSLQPLALEGSLQ

(19) United States
(12) Patent Application Publication
(10) Pub. No.: US 2025/0197911 A1
(43) Pub. Date: Jun. 19, 2025

(54) Production of Hybrid Peptides by Antigen Presenting Cells

(71) Applicant: Joslin Diabetes Center, Boston, MA (US)

(72) Inventor: Jason Gaglia, Boston, MA (US)

(73) Assignee: Joslin Diabetes Center, Boston, MA (US)

(21) Appl. No.: 18/961,894

(22) Filed: Nov. 27, 2024

Related U.S. Application Data
(63) Continuation of application No. PCT/US23/67632, filed on May 30, 2023.
(60) Provisional application No. 63/347,257, filed on May 31, 2022.

(51) Int. Cl. C12P 21/02; A61K 40/24; A61K 40/34; A61K 40/41; C07K 14/62; G01N 33/50

(52) U.S. Cl. CPC C12P 21/02; A61K 40/24; A61K 40/34; A61K 40/416; C07K 14/62; G01N 33/505; A61K 2239/39; G01N 2333/62

(57) ABSTRACT

This disclosure describes production of hybrid peptides, including hybrid insulin peptides (HIPs), by antigen presenting cells (APCs).

This application is a continuation of International Application No. PCT/US2023/067632, filed May 30, 2023, which claims priority to U.S. Provisional Application No. 63/347,257, filed May 31, 2022, which are incorporated herein in their entirety for any purpose.

### REFERENCE TO SEQUENCE LISTING

The present application contains a Sequence Listing which has been submitted electronically in XML format. Said XML copy, created on May 26, 2023, is named “2023 May 26-01123-0013-00PCT-ST26” and is 51,081 bytes in size. The information in the electronic format of the sequence listing is incorporated herein by reference in its entirety.

### FIELD

This disclosure describes production of hybrid peptides, including hybrid insulin peptides (HIPs), by antigen presenting cells (APCs).

### BACKGROUND

Type 1 Diabetes (T1D) is an autoimmune disease characterized by selective loss of pancreatic β-cells. Aberrant reactivity to insulin and insulin precursor peptides has long been considered important in the pathogenesis of T1D. Recently, T cells recognizing hybrid insulin peptides (HIPs) have been described, first in a rodent model and later in human T1D. A HIP is a CD4 T-cell epitope that is formed by the post-translational fusion of two peptide fragments with at least one of the peptide fragments derived from insulin or a precursor. HIP formation can occur intra- or inter-molecularly. The role of these HIPs in the pathogenesis of T1D remains unclear. Some have proposed that HIPs are formed in β-cells during crinophagy of secretory granules. As ultrastructural studies have demonstrated that β-cells can transfer secretory vesicles to local antigen presenting cells (APCs), the present study evaluated whether APCs could similarly generate HIPs via protease mediated transpeptidation. Peptide-containing nanocarriers (i.e., pseudogranules) were prepared mimicking β-cell secretory vesicles to transfer selected peptides to APCs in both mouse and human in vitro systems utilizing donor APCs and CD4 TCR transgenic T cells or a CD4 transductant reporter cells, respectively. Data indicate that different subsets of APCs are capable of intramolecular and intermolecular HIP formation, and HIP formation can be blocked by proteosome inhibition.

In the present study, nanocarriers were used to prepare pseudogranules comprising synthetic peptides for simulation of beta cell granules, with a nanocarrier size of 200-500 nm in diameter. These pseudogranules were taken up by APCs, leading to formation of hybrid proteins, such as HIPs, via proteosome activity. This allows for presentation of hybrid proteins to T cells. Nanocarriers may be biodegradable, as described in Shen et al., Immunology 117(1): 78-88 (2006).

Using the pseudogranules described herein, a variety of APCs were able to generate HIPs. Thus, APCs may play a role in the processing and presentation of CD4-specific neoepitopes in patients with type 1 diabetes. This disclosures also identifies a mechanism for the generation of HIPs recognized in T1D by APCs.

### SUMMARY

In some embodiments, a method of preparing hybrid peptides in antigen presenting cells comprises encapsulating at least two synthetic peptides or a protein within nanocarriers, incubating the nanocarriers with an antigen presenting cell (APC), wherein incubation results in the fusion of two or more peptides or portions of one protein to produce a hybrid peptide.

In some embodiments, all or part of the hybrid peptide is presented on the APC surface.

In some embodiments, the hybrid peptide comprises two or more fragments of the same peptide or protein.

In some embodiments, the hybrid peptide comprises two or more fragments of different peptides or proteins.

In some embodiments, at least one peptide or a protein is insulin, a fragment of insulin, a precursor of insulin, or a fragment of a precursor of insulin, and wherein a hybrid insulin peptide (HIP) is formed.

In some embodiments, the nanocarrier is 200-500 nm in diameter.

In some embodiments, the antigen presenting cell is a monocyte, dendritic cell, macrophage, B cell, or T cell.

In some embodiments, a method of inhibiting hybrid peptide production comprises treating a sample comprising an APC and a T cell with one or more proteosome inhibitor.

In some embodiments, inhibiting hybrid peptide production leads to a decrease in T-cell activation.

In some embodiments, a method of treating a patient with an immune disorder comprises administering one or more proteosome inhibitor, wherein the administering leads to inhibiting hybrid peptide production by APCs.

In some embodiments, inhibiting hybrid peptide production leads to a decrease in T-cell activation.

In some embodiments, a method of evaluating an immune response in a sample comprising an APC comprises encapsulating at least two synthetic peptides or a protein within nanocarriers; incubating the nanocarriers with the sample, wherein incubation results in the fusion of two or more peptides or portions of a protein to produce an APC presenting one or more hybrid peptides on the APC surface; and measuring an immune response to one or more hybrid peptides on the APC surface with a reporter assay.

In some embodiments, the reporter assay measures T-cell activation.

In some embodiments, the reporter assay uses hybridomas expressing CD4.

In some embodiments, the reporter assay measures interferon gamma levels.

In some embodiments, the reporter assay measures interleukin-10 levels.

In some embodiments, the hybrid peptide is a hybrid insulin peptide comprising insulin, a fragment of insulin, a precursor of insulin, or a fragment of a precursor of insulin.

### BRIEF DESCRIPTION OF THE DRAWINGS

FIG. 1 shows hybrid peptide production by antigen presenting cells (APCs). Synthetic peptides or proteins are encapsulated within nanocarriers, and these nanocarriers are then fed to APCs. The APCs can then form hybrid peptides from the peptides or proteins within the nanocarriers. The nanocarrier may allow for preparation of hybrid peptides by APCs by holding different peptides or proteins in close proximity. Hybrid peptides may be generated from two copies of the same protein (intramolecular spliced peptides) or from two different proteins (intermolecular spliced peptides). The leftward slashes and rightward slashes in the intramolecular spliced peptides indicate that amino acid sequence from two separate copies of the same peptides (for example, two copies of insulin) were fused into the hybrid peptide. An intermolecular spliced peptide can be generated from copies of two different peptides (for example, insulin and a different peptide). These hybrid peptides can exist in an APC together with linear proteins (i.e., non-hybrid peptides, which are not spliced).

FIG. 2 shows intermolecular BDC2.5 HIP formation in murine APCs.

FIG. 3 shows that APCs stimulate HIP11 reactive T cells. HIP11 is a C-peptide/C-peptide HIP. Human PBMCs were stained with anti-CD3 antibody and magnetically sorted to deplete T cells. Pseudogranules were added to flowthrough and co-cultured with T-cell hybridoma cells (as described in Mann et al., Front Immunol 9 (11): 633 (2020)). The CD4 hybridoma cells have a ZsGreen reporter that can be activated (linked to NFAT), and the cells are HLA-DQ8 specific and reactive for HIP11. Flow cytometry analysis of TCR reporter was performed to measure T-cell stimulation.

FIGS. 4A-4F shows results with a variety of different APCs, including B cells (A), APCs without B cells (B), T cells (C), monocytes (D), dendritic cells (E), and macrophages (F).

FIG. 5 shows inhibition of HIP11 production (as measured by interferon gamma levels) by various proteosome inhibitors.

FIGS. 6A and 6B show inhibition of BDC2.5 recognized peptide production (as measured by interferon gamma levels) by various proteosome inhibitors at higher (A) and lower (B) concentrations.

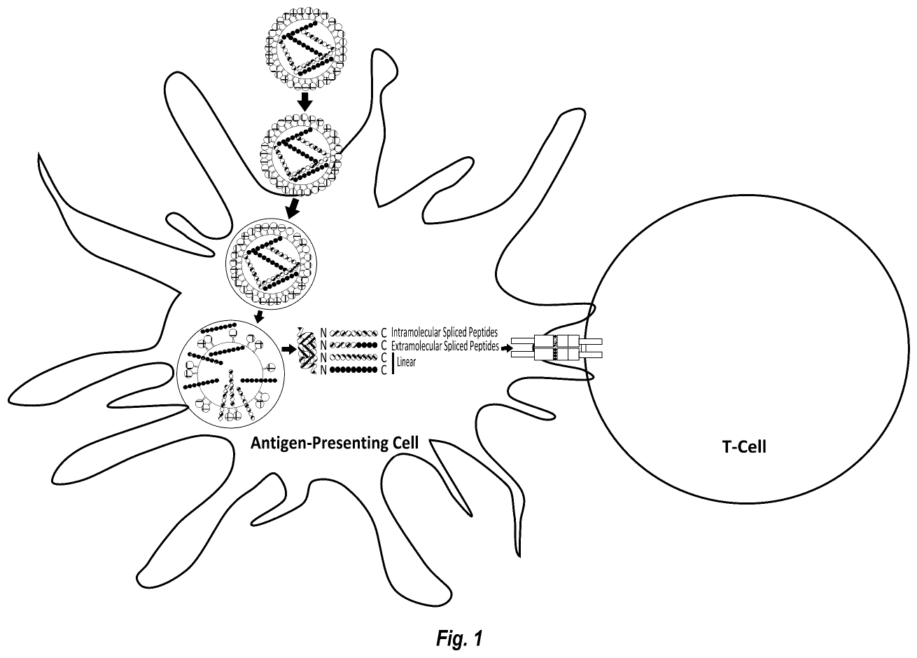

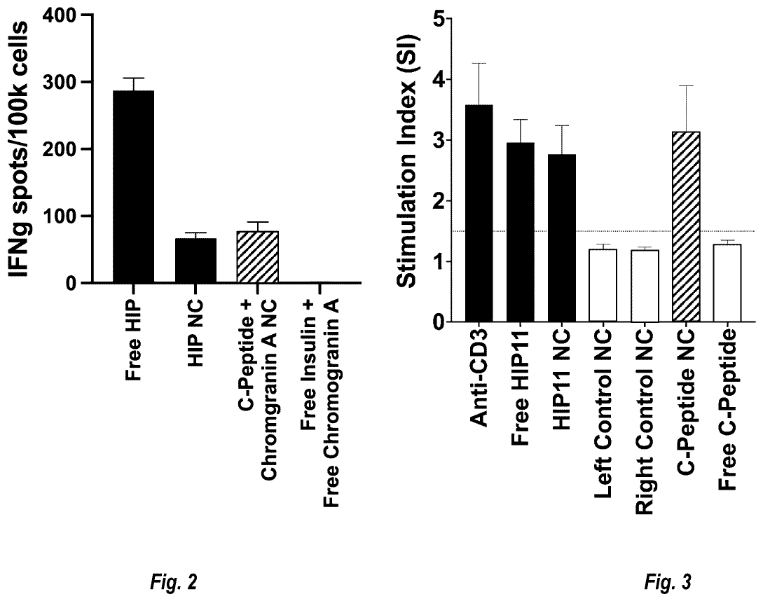

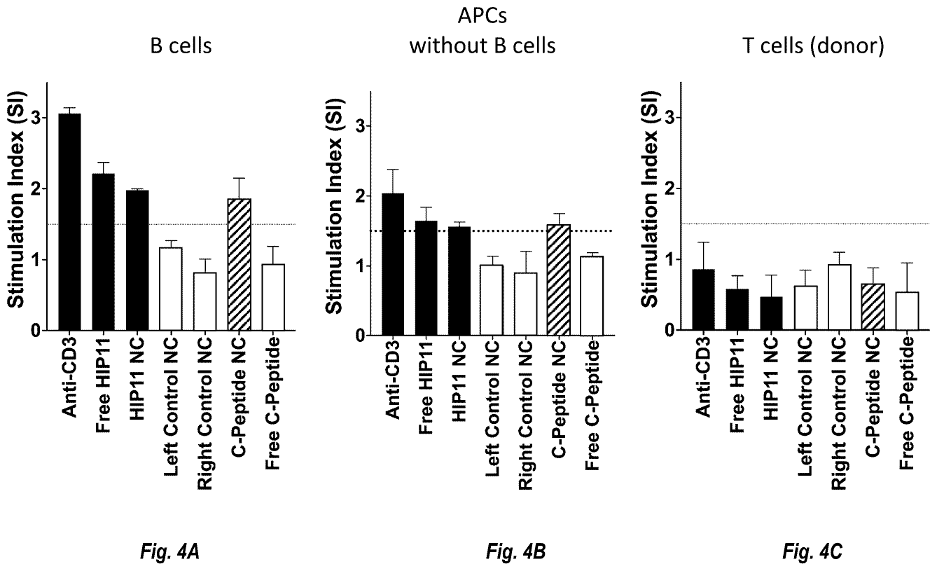

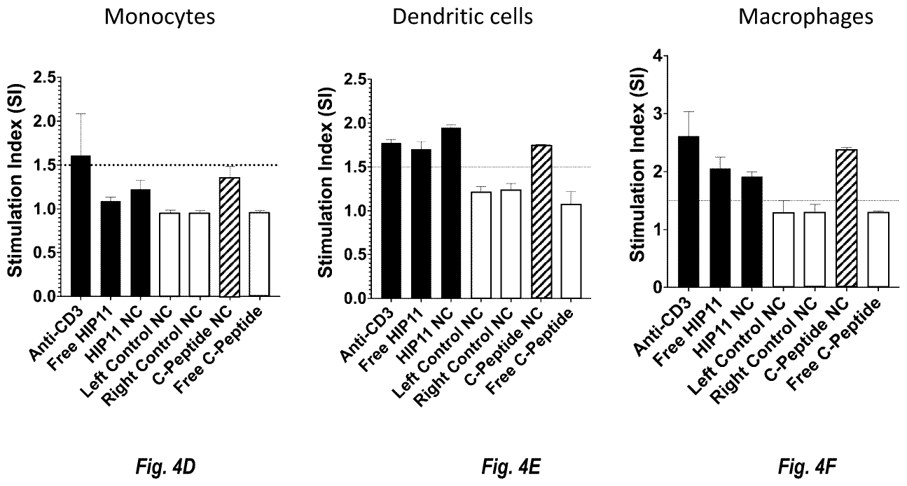

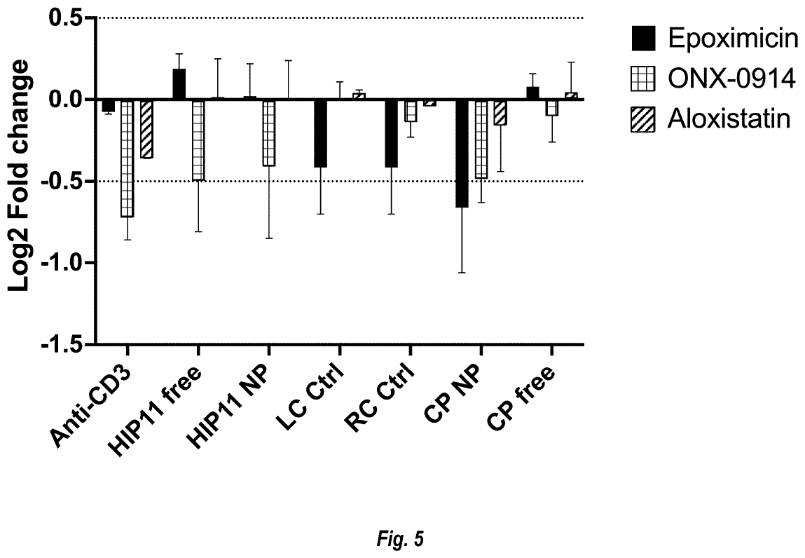

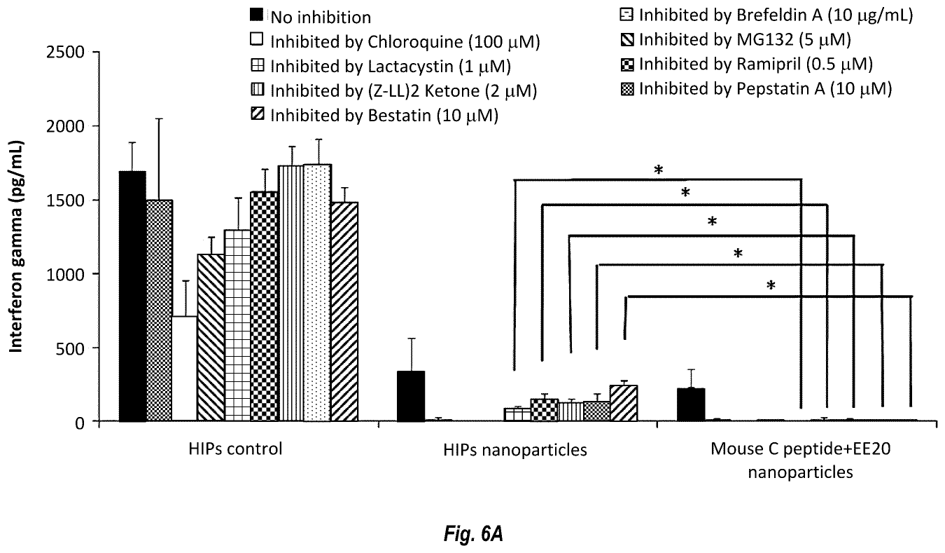

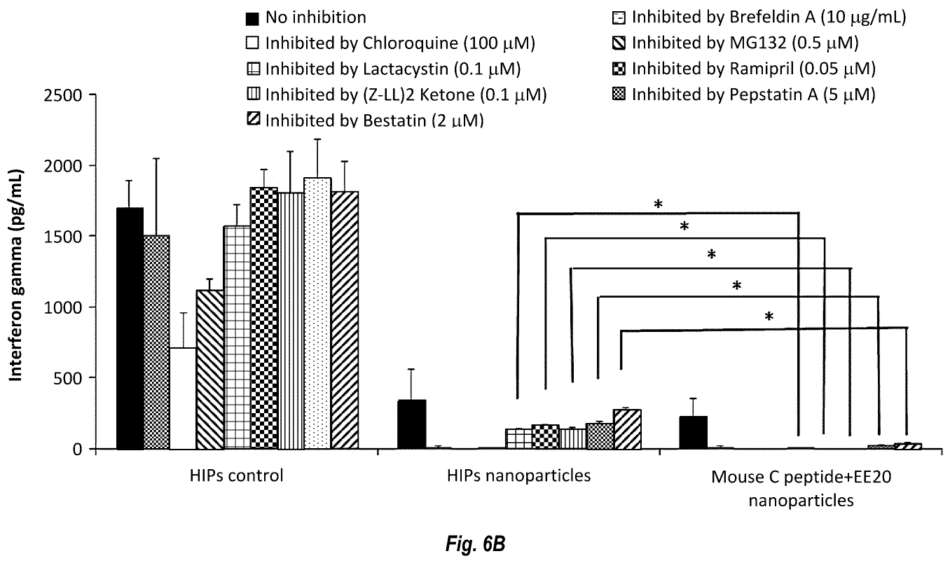

### DESCRIPTION OF THE SEQUENCES

Table 1 provides a listing of certain sequences referenced herein. Hyphenation indicates hybrid peptide junction. Bold sequences represent C-peptide components of HIPs. Italicized sequences represent N-terminal sequences of natural cleavage products.

TABLE 1
Description of the Sequences

| | | SEQ |
|---|---|---|
| | | ID |
| Description | Sequences | NO |
| BDC2.5 HIP: | LQTLAL-WSRMD | 1 |
| C-Peptide/ | | |
| ChrA-WE14 | | |
| Chromogranin A | WSRMDQLAKELTAE | 2 |
| WE14 peptide | | |
| C-Peptide | MALWMRFLPLLALLFLWESHPT | 3 |
| peptide | QAFVKQHLCGSHLVEALYLVCG | |
| (mouse) | ERGFFYTPMSRREVEDPQVAQL | |
| | ELGGGPGAGDLQTLALEVAQQK | |
| | RGIVDQCCTSICSLYQLENYCN | |
| Human HIP11 | SLQPLAL-EAEDLQV | 4 |
| C-peptide | EAEDLQVGQVELGGGPGAGSLQ | 5 |
| peptide | PLALEGSLQ | |
| (human) | | |
| C-terminal | SLQPLAL | 6 |
| sequence from | | |
| HIP11 | | |
| N-terminal | EAEDLQV | 7 |
| sequence from | | |
| HIP11 | | |
| HIP1 | GQVELGG-WSKMDQLA | 8 |
| HIP2 | GQVELGG-LEGQEEEE | 9 |
| HIP3 | GQVELGGG-EAEDLQV | 10 |
| HIP4 | GQVELGGG-GIVEQCC | 11 |
| HIP5 | GQVELGGG-TPIESHQ | 12 |
| HIP6 | GQVELGGG-NAVEVLK | 13 |
| HIP7 | GQVELGGG-FLGEGHH | 14 |
| HIP8 | GQVELGGG-SSPETLI | 15 |
| HIP9 | SLQPLAL-WSKMDQL | 16 |
| HIP10 | SLQPLAL-LEGQEEE | 17 |
| HIP12 | SLQPLAL-GIVEQCC | 18 |
| HIP13 | SLQPLAL-TPIESHQ | 19 |
| HIP14 | SLQPLAL-NAVEVLK | 20 |
| HIP15 | SLQPLAL-FLGEGHH | 21 |
| HIP16 | SLQPLAL-SSPETLI | 22 |
| insB: | SHLVEALYLVCGER | 23 |
| 9-23 | | |
| insB: | SHLVEALYLVCGEE | 24 |
| 9-23R22E | | |
| ins₆₄₋₇₉ | GQVELGGGPGAGSLQP | 25 |
| ins₇₅₋₉₀ | GSLQPLALEGSLQKRG | 26 |
| ChgA₃₃₄₋₃₄₉ | KEWEDSKRWSKMDQLA | 27 |
| ChgA₃₅₀₋₃₆₅ | KELTAEKRLEGQEEEE | 28 |
| Ins₄₉₋₆₄ | FYTPKTRREAEDLQVG | 29 |
| Ins₈₂₋₉₇ | LEGSLQKRGIVEQCCT | 30 |
| IAPP₁₅₋₃₀ | VALNHLKATPIESHQV | 31 |
| IAPP₆₆₋₈₁ | GSNTYGKRNAVEVLKR | 32 |
| ScG1₄₃₂₋₄₄₇ | SDTREEKRFLGEGHHR | 33 |
| NP-Y₆₀₋₇₅ | TRQRYGKRSSPETLIS | 34 |
| Insulin specific | EAEDLQVGQVELGGGPGAGS | 35 |
| peptide | | |
| Insulin specific | GSLQPLALEGSLQ | 36 |
| peptide | | |
| Insulin specific | GPGAGSLQPLALEGSLQ | 37 |
| peptide | | |
| Insulin specific | EAEDLQVGQVELGGGPGAGS | 38 |
| peptide | | |
| Insulin specific | EAEDLQVGQVELGGGPGAGS | 39 |
| peptide | LQ | |
| Insulin specific | GGGPGAGSLQPLALEGSLQ | 40 |
| peptide | | |

A number of human HIP sequences have been presented in Baker et al. Diabetes 68 (9): 1830-1840 (2019), as shown in the Table 2 below.

TABLE 2
Additional representative human HIPS

| | SEQ | | | SEQ | |
|---|---|---|---|---|---|
| | ID | | | ID | |
| HIPS | NO | Peptide Sequence* | B-Chain | NO | Peptide Sequence |
| HIP1 | 8 | GQVELGG-WSKMDQLA | insB: | 23 | SHLVEALYLVCGER |
| | | | 9-23 | | |
| HIP2 | 9 | GQVELGG-LEGQEEEE | insB: | 24 | SHLVEALYLVCGEE |
| | | | 9-23R22E | | |
| HIP3 | 10 | GQVELGGG-EAEDLQV | | | |
| HIP4 | 11 | GQVELGGG-GIVEQCC | Left | | Peptide Sequence** |
| | | | control | | |
| | | | peptides | | |
| HIP5 | 12 | GQVELGGG-TPIESHQ | ins₆₄₋₇₉ | 25 | GQVELGGGPGAGSLQP |
| HIP6 | 13 | GQVELGGG-NAVEVLK | ins₇₅₋₉₀ | 26 | GSLQPLALEGSLQKRG |
| HIP7 | 14 | GQVELGGG-FLGEGHH | | | |
| HIP8 | 15 | GQVELGGG-SSPETLI | Right | | Peptide Sequence |
| | | | control | | |
| | | | peptides | | |
| HIP9 | 16 | SLQPLAL-WSKMDQL | ChgA₃₃₄₋₃₄₉ | 27 | KEWEDSKRWSKMDQLA |
| HIP10 | 17 | SLQPLAL-LEGQEEE | ChgA₃₅₀₋₃₆₅ | 28 | KELTAEKRLEGQEEEE |
| HIP11 | 5 | SLQPLAL-EAEDLQV | Ins₄₉₋₆₄ | 29 | FYTPKTRREAEDLQVG |
| HIP12 | 18 | SLQPLAL-GIVEQCC | Ins₈₂₋₉₇ | 30 | LEGSLQKRGIVEQCCT |
| HIP13 | 19 | SLQPLAL-TPIESHQ | IAPP₁₅₋₃₀ | 31 | VALNHLKATPIESHQV |
| HIP14 | 20 | SLQPLAL-NAVEVLK | IAPP₆₆₋₈₁ | 32 | GSNTYGKRNAVEVLKR |
| HIP15 | 21 | SLQPLAL-FLGEGHH | SCG1₄₃₂₋₄₄₇ | 33 | SDTREEKRFLGEGHHR |
| HIP16 | 22 | SLQPLAL-SSPETLI | NP-Y₆₀₋₇₅ | 34 | TRQRYGKRSSPETLIS |

*Hyphenation indicates hybrid peptide junction.
**Bold sequences represent C-peptide components of HIPs.
***Italicized sequences represent N-terminal sequences of natural cleavage products.

Mass spectrometry data are shown in Table 3.

TABLE 3
Mass spectrometry data

| Sample ID | Reference | Peptide Sequences |
|---|---|---|
| Positive | HIP11 | SLQPLAL-EAEDLQV |
| Control | | (SEQ ID NO: 4) |
| (HIP11 | | |
| peptide) | | |
| HIP11 | HIP11 | SLQPLAL-EAEDLQV |
| Nanocarrier | | (SEQ ID NO: 4) |
| C-Peptide | HIP11 | SLQPLAL-EAEDLQV |
| Nanocarrier | | (SEQ ID NO: 4) |
| | Insulin | EAEDLQVGQVELGGGPGAGS |
| | specific | (SEQ ID NO: 35) |
| | Peptides | GSLQPLALEGSLQ |
| | | (SEQ ID NO: 36) |
| | | GPGAGSLQPLALEGSLQ |
| | | (SEQ ID NO: 37) |
| PPI | HIP11 | SLQPLAL-EAEDLQV |
| Nanocarrier | | (SEQ ID NO: 4) |
| | Insulin | EAEDLQVGQVELGGGPGAGS |
| | specific | (SEQ ID NO: 38) |
| | Peptides | EAEDLQVGQVELGGGPGAGSLQ |
| | | (SEQ ID NO: 39) |
| | | GGGPGAGSLQPLALEGSLQ |
| | | (SEQ ID NO: 40) |

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method of preparing hybrid peptides in antigen presenting cells comprising:
a. encapsulating at least two synthetic peptides or a protein within nanocarriers,
b. incubating the nanocarriers with an antigen presenting cell (APC), wherein incubation results in the fusion of two or more peptides or portions of one protein to produce a hybrid peptide.

2. The method of claim 1, wherein all or part of the hybrid peptide is presented on the APC surface.

3. The method of claim 1, wherein the hybrid peptide comprises two or more fragments of the same peptide or protein.

4. The method of claim 1, wherein the hybrid peptide comprises two or more fragments of different peptides or proteins.

5. The method of claim 1, wherein at least one peptide or a protein is insulin, a fragment of insulin, a precursor of insulin, or a fragment of a precursor of insulin, and wherein a hybrid insulin peptide (HIP) is formed.

6. The method of claim 1, wherein the nanocarrier is 200-500 nm in diameter.

7. The method of claim 1, wherein the antigen presenting cell is a monocyte, dendritic cell, macrophage, B cell, or T cell.

8. (canceled)

9. (canceled)

10. A method of treating a patient with an immune disorder comprising administering one or more proteosome inhibitor, wherein the administering leads to inhibiting hybrid peptide production by APCs.

11. The method of claim 10, wherein inhibiting hybrid peptide production leads to a decrease in T-cell activation.

12. A method of evaluating an immune response in a sample comprising an APC comprising:
a. encapsulating at least two synthetic peptides or a protein within nanocarriers;
b. incubating the nanocarriers with the sample, wherein incubation results in the fusion of two or more peptides or portions of a protein to produce an APC presenting one or more hybrid peptides on the APC surface; and
c. measuring an immune response to one or more hybrid peptides on the APC surface with a reporter assay.

13. The method of claim 12, wherein the reporter assay measures T-cell activation.

14. The method of claim 13, wherein the reporter assay uses hybridomas expressing CD4.

15. The method of claim 12, wherein the reporter assay measures interferon gamma levels.

16. The method of claim 12, wherein the reporter assay measures interleukin-10 levels.

17. The method of claim 12, wherein the hybrid peptide is a hybrid insulin peptide comprising insulin, a fragment of insulin, a precursor of insulin, or a fragment of a precursor of insulin.
